(19) United States
(12) Patent Application Publication
(10) Pub. No.: US 2025/0302973 A1
(43) Pub. Date: Oct. 2, 2025

(54) POLYMER CONJUGATES OF DRUGS WITH CENTRAL NERVOUS SYSTEM (CNS) EFFECTS AND PERIPHERAL NMDAR BLOCKING ACTIVITY AND/OR IMMUNE SYSTEM MODULATING EFFECTS

(71) Applicants: UNIVERSITY OF PADOVA, Padua (IT); MGGM LLC, Kerhonkson, NY (US)

(72) Inventors: Paolo L. Manfredi, Miami Beach, FL (US); Charles E. Inturrisi, New York, NY (US); Andrea Mattarei, Perarolo di Vigonza (IT); Marco Banzato, Galzignano Terme (IT); Gianfranco Pasut, Padova (IT); Sara De Martin, Vigonza (IT); Stefano Comai, Trento (IT)

(21) Appl. No.: 18/864,924

(22) PCT Filed: May 12, 2023
(86) PCT No.: PCT/US2023/022058

§ 371 Date: Nov. 12, 2024

Related U.S. Application Data
(60) Provisional application No. 63/341,198, filed on May 12, 2022.

(51) Int. Cl. A61K 47/60; A61K 9/00; A61P 11/00; A61P 29/00

(52) U.S. Cl. CPC A61K 47/60; A61K 9/0019; A61P 11/00; A61P 29/00

(57) ABSTRACT

The present invention discloses novel molecules consisting of N-methyl-D-aspartate receptor (NMDAR) antagonists-polymer conjugates having a general structure D-(X-Poly-T)n, wherein D is an high affinity, i.e., (+)-, (−)-, or (±)-dizocilpine, or a low affinity, i.e., (+)-, (−)-, or (±)-methadone, CNS active NMDAR antagonist, n is an integer comprised between 1 and 6. X is a stable (enzymatically and/or hydrolytically under physiological conditions) linker comprising a covalent bond or a chain of atoms that covalently attaches a small molecule NMDAR antagonist drug moiety to the Poly derivative. Poly is a covalently bonded chain of repeating monomer units that form a polymer or an oligomer backbone of synthetic or natural origin. T, if present, is either another molecule of D, or a terminal group of Poly, represented by any suitable chemical group which, depending upon preference, is unreactive or reactive with other chemical moieties, or has a targeting property.

### CROSS-REFERENCE TO RELATED APPLICATION

This application claims priority to, and the benefit of the filing date of, U.S. Patent Application Ser. No. 63/341,198, filed on May 12, 2022, the disclosure of which is incorporated by reference herein in its entirety.

### FIELD OF THE INVENTION

Aspects of the present invention generally relate to polymer conjugates of N-methyl-D-aspartate receptor (NMDAR) antagonists, and therapeutic and preventative aspects of same on the respiratory system and inflammation.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The NMDAR is a ionotropic receptor that requires the binding of glutamate (the primary excitatory neurotransmitter in the human brain), glycin, and a voltage dependend Mg²⁺ disengagement before allowing a subtype-specific tightly regulated influx of Ca²⁺ [Hansen K B, Yi F, Perszyk R E, et al. Structure, function, and allosteric modulation of NMDA receptors. J Gen Physiol. 2018; 150(8):1081-1105. doi:10.1085/jgp.201812032]. In the brain, the NMDAR plays a role in synaptic plasticity, which is a neuronal mechanism at the basis of memory formation. Excessive NMDAR activity has been linked to excitotoxicity, a toxic cellular state caused by excessive Ca²⁺ influx that can lead to impairment of neural plasticity and cell death. Many investigational and approved drugs are known to antagonize the activity of NMDARs, including MK-801, ketamine, dextromethorphan, and esmethadone (dextromethadone), by binding to the receptor within the NMDAR pore with different affinity. Apart from their role in the CNS, NMDARs are also heavily expressed in peripheral (extra-CNS) tissues (Du J, Li X H, Li Y J. Glutamate in peripheral organs: Biology and pharmacology. Eur J Pharmacol. 2016; 784:42-48. doi:10.1016/j.ejphar.2016.05.009). NMDAR-modulating drugs may therefore also therapeutically target extra CNS receptors, with downstream effects that have potential therapeutic value against dysregulated Ca²⁺ and or inflammation. The inventors previously disclosed potential peripheral (out of the CNS) therapeutic effects of NMDAR antagonists in U.S. Patent Application Publication No. 2023/0017786. However, NMDAR antagonist drugs that cross the blood brain barrier (BBB)—such as those in that previous application—also have central nervous system effects. Therefore, preferential targeting of tperipheral NMDARs by high- and low-affinity antagonists with restricted access to the CNS is a potential novel strategy to exert peripheral NMDAR modulating therapeutic effects while avoiding CNS effects, including psychoactive effects, including dissociative effects or hallucinatory effects, and citotoxic effects, including Olney's lesions [Olney, J. W., Labruyere, J., and Price, M. T. (1989). Pathological Changes Induced in Cerebrocortical Neurons by Phencyclidine and Related Drugs. Science 244, 1360-1362. Doi:10.1126/science.2660263; Olney, J. W., Labruyere, J., Wang, G., Wozniak, D. F., Price, M. T., and Sesma, M. A. (1991). NMDA Antagonist Neurotoxicity: Mechanism and Prevention. Science 254, 1515-1518. Doi:10.1126/science.1835799; Fix, A. S., Horn, J. W., Wightman, K. A., Johnson, C. A., Long, G. G., Storts, R. W., et al. (1993). Neuronal Vacuolization and Necrosis Induced by the Noncompetitive N-Methyl-D-Aspartate (NMDA) Antagonist MK(+)801(Dizocilpine Maleate): a Light and Electron Microscopic Evaluation of the Rat Retrosplenial Cortex. Exp. Neurol. 123, 204-215. Doi:10.1006/exnr.1993. 1153].

CNS pychoactive drugs cross the BBB and reach receptors in the brain, including NMDARs, and exert certain central nervous system effects, including therapeutic and side including potentially toxic central nervous system effects. The central nervous system effects of NMDARs antagonists are primarily caused by their binding to NMDARs located within the membrane of neurons in the brain. NMDARs are heterotetramers formed of subunits from three groups of genes, named GluN1, GluN2, and GluN3. Like the GluN1 subunits, the GluN3 subunits, coded by two different genes (A-B), bind the co-agonists glycine or d-serine, while GluN2 subunits, encoded by four different genes (A-D), bind glutamate or NMDA. NMDARs cannot form functional homotetramers. The obligatory heterotetramers can consist of a wide variety of subunit combinations and confer functional diversity. Typically this includes two GluN1 subunits and either two GluN2 subunits of the same or different subtypes, or two GluN1 subunits and a GluN2 and a GluN3 subunit.

Furthermore, NMDARs with different subunit compositions show spatio-temporal variation, with GluN2B and GluN2D expression highest in early development, shifting to increased, but not exclusive, GluN2A and GluN2C expression later in life, with expression levels varying across different regions of the brain. Drugs acting as NMDAR antagonists may have prominent therapeutic psychoactive effects, including therapeutic effects such as antidepressant effects. However, NMDAR uncompetitive antagonists, such as for example MK-801 and ketamine, with high affinity for NMDARs cause dissociative effects even when given at potentially therapeutic doses. NMDAR antagonists are approved or under clinical investigation for a multiplicity of psychiatric or neurodegenerative diseases and symptoms, including derpression (esketamine, arketamine, ketamine, esmethadone, dextomethorphan), Alzheimer disease (memantine), Parkinson disease (amantadine), or for the induction of anesthesia, procedural sedation, and analgesia (ketamine). However, some NMDAR antagonists may have prominent central nervous system effects, including dissociative effects (MK-801, ketamine and esketamine) and these effects impede their development as potentially therapeutic drugs. There are strong public safety and regulatory concerns about the therapeutic uses of substances with the potential for inducing dissociative effects. In summary, the development of psychoactive substances for the treatment of diseases, disorders and conditions, and symptoms, including extra-CNS diseases, disorders, conditions, and symptoms remains problematic due to the potent central nervous system effects of these drugs, which currently can be modulated only by dose reduction.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention discloses NMDAR antagonist polymer conjugates with therapeutic advantages over known NMDAR antagonists. The chemically modified drugs described herein have applications in the fields of drug discovery and pharmacotherapy, polymer chemistry, and others. Of particular interest are therapeutic and preventive effects of these new molecular entities on the immune system, the respiratory system, the digestive system, the genitourinary system, and the cardiovascular system.

The aim to decrease CNS side effects from NMDAR uncompetitive antagonists is pursued in the present invention by decreasing the ability of these drugs to cross (1) the BBB, an anatomic-functional barrier effective at eliminating or reducing the passage of the different molecules into the brain, and/or (2) the intestinal barrier (IB) an anatomic-functional barrier effective at eliminating or reducing the passage of the different molecules across the digestive system. To accomplish this, the present inventors designed polymer-drug conjugates (PDCs) of NMDAR antagonists to impede, decrease, or modulate their crossing of the BBB. For certain compounds, there may also be an advantage in modulating access across the intestinal barrier (IB). Potentially therapeutic peripheric effects of these conjugates could become more advantageous in the absence or with reduced BBB and IB crossing, leading to concomitant down-modulation of CNS effects or even restricting the drug to the gastrointestinal tract. In fact, the molecules disclosed in this application cannot cross the BBB or have modulated or limited BBB crossing abilities, because of specific features of the polymer structure, their molecular weight, and/or their hydrodynamic volume and their chemical-physical properties. Their coupling with a specific tailored polymer may result in novel molecules with improved pharmacokinetic and pharmacodynamic profile for select diseases and disorders a favorable risk-benefit ratio. In summary, this invention provides PDCs with the intent and ability to preferentially target NMDARs located outside the CNS for the treatment of diseases, disorders and conditions linked to unbalanced activity and or excitotoxicty due to peripheral NMDARs dysregulation and or immune system dysfunction.

Aspects of the present invention are directed to NMDAR antagonist polymer conjugates having a general structure D-(X-Poly-T)n, wherein D is a CNS active NMDAR antagonist and n is an integer comprised between 1 and 6.

X is a stable (enzymatically and/or hydrolytically under physiological conditions) linker comprising a covalent bond or a chain of atoms that covalently attaches a small molecule NMDA antagonist drug moiety to the Poly derivative. The drug moiety has at least one chemically reactive functional group (e.g. a primary amine or secondary amine, hydroxyl, sulfhydryl, carboxyl, aldehyde or ketone), or if absent this group can be chemically introduced, pendant thereto chemically reacted to the linker to form a covalent bond. Examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, thioether.

Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, mPEG-O-559 Da, where “m” means methoxy.

T, if present, is either D or a terminal group of the Poly, when T is a terminal group it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, Nsuccinimidyloxy, sulfo-N-succinimidyloxy, 1-benzotriazol, 1-imidazolyloxy, p-nitrophenyloxy, formyl. We have begun synthesizing PEG derivatives of esmethadone by connecting a short PEG chain (oligo(ethylene glycol)) to one of the phenyl functionalities of esmethadone. We selected the phenyl rings for PEG conjugation because of our previous work on esmethadone analogues suggesting that modifications at the level of the phenyl ring caused the least hindrance to NMDAR activity.

In one particular embodiment, the present invention includes a compound including a structural analogue to (R)-methadone ((−)-methadone, levomethadone), in free base form, and/or in a pharmaceutical acceptable salt form thereof according to formula I:

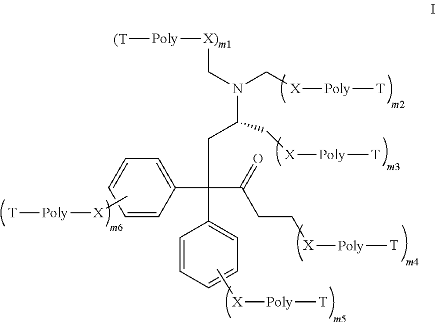

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

In another particular embodiment, the present invention includes a compound including a structural analogue to (S)-methadone ((+)-methadone, dextromethadone, esmethadone), in free base form, and/or in a pharmaceutical acceptable salt form thereof, according to formula II:

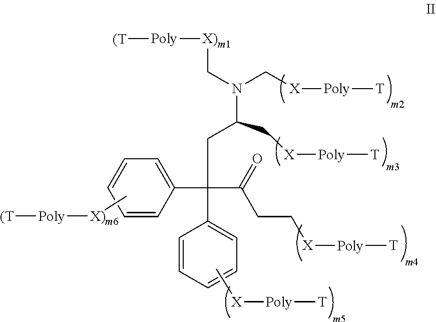

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

In another particular embodiment, the present invention includes a compound having a structural analogue to (S, R)-methadone ((±)-methadone, rac-methadone, methadone), in free base form, and/or in pharmaceutical acceptable salt form thereof according to formula III:

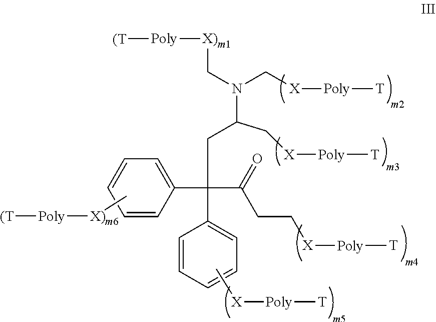

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

In another particular embodiment, the present invention includes a compound having a structural analogue to (−)-dizocilpine ((−)-MK-801) in free base form, and/or in pharmaceutical acceptable salt form thereof according to formula IV:

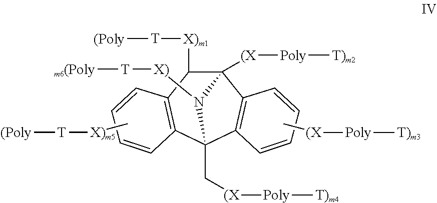

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

In another particular embodiment, the present invention includes a compound having a structural analogue to (+)-dizocilpine ((+)-MK-801) in free base form, and/or in pharmaceutical acceptable salt form thereof according to formula V:

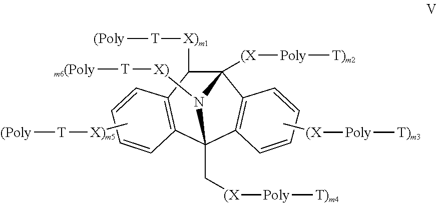

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

In another particular embodiment, the present invention includes a compound having a structural analogue to (±)-dizocilpine ((±)-MK-801) in free base form, and/or in pharmaceutical acceptable salt form thereof according to formula VI:

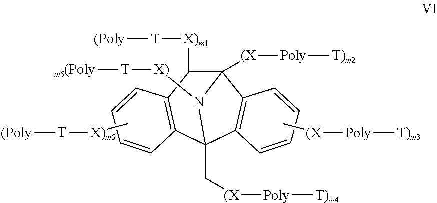

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative;
Poly is a covalently bonded chain of repeating monomer units that form a polymer backbone of synthetic or natural origin; and
T, if present, is either (−)-methadone or a terminal group of Poly, (and “X,” “Poly,” and “T” may be as described above).

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, explain the principles of the present invention.

FIGS. 1A-1B are graphs showing the effect of the NMDAR antagonists esmethadone and MK-801 on the mRNA expression of inflammatory cytokines in A-549 cells in two different experimental setups. The effect of the tested NMDAR antagonists on pulmonary cells is mediated by macrophages. *P<0.05, **P<0.01, ***P<0.001.

FIG. 2 is a graph showing the effects of 0.3 mg/kg MK-801 and MK-801-EGME conjugates at the equivalent dose of 0.3 mg/kg MK-801 on the distance travelled by mice during 10 min in the Open Field Test (n=7 mice per group). Data are reported as mean±SEM. **** P<0.0001 vs. Vehicle; ####P<0.0001 vs. MK-801 by one-way ANOVA followed by Bonferroni post-hoc multiple comparisons.

FIG. 3 is a schematic showing the synthesis of (+)-MK-801-EGME conjugates using the strategy proposed in this specification.

FIG. 4 is a graph showing the ¹H NMR spectrum of (5S,10R)-5-methyl-12-(2,5,8,11-tetraoxatridecan-13-yl)-10,11-dihydro-5H-5,10-epiminodibenzo[a,d][7]annulene hydrochloride ((+)-MK-801-TetraEGME x HCl).

FIG. 5 is a graph showing the ¹³C NMR spectrum of (5S,10R)-5-methyl-12-(2,5,8,11-tetraoxatridecan-13-yl)-10,11-dihydro-5H-5,10-epiminodibenzo[a,d][7]annulene hydrochloride ((+)-MK-801-TetraEGME x HCl).

FIG. 6 is a graph showing the ¹H NMR spectrum of (5S,10R)-5-methyl-12-(2,5,8,11,14,17-hexaoxanonadecan-19-yl)-10,11-dihydro-5H-5,10-epiminodibenzo[a,d][7]annulene hydrochloride ((+)-MK-801-HexaEGME x HCQ).

FIG. 7 is a graph showing the ¹³C NMR spectrum of (5S,10R)-5-methyl-12-(2,5,8,11,14,17-hexaoxanonadecan-19-yl)-10,11-dihydro-5H-5,10-epiminodibenzo[a,d][7]annulene hydrochloride ((+)-MK-801-HexaEGME x HCl).

FIG. 8 is a graph showing the ¹H NMR spectrum of (5S,10R)-5-methyl-12-(2,5,8,11,14,17,20,23-octaoxapentacosan-25-yl)-10,11-dihydro-5H-5,10-epiminodibenzo[a,d][7]annulene hydrochloride ((+)-MK-801-OctaEGME x HCl).

FIG. 9 is a graph showing the ¹³C NMR spectrum of (5S,10R)-5-methyl-12-(2,5,8,11,14,17,20,23-octaoxapentacosan-25-yl)-10,11-dihydro-5H-5,10-epiminodibenzo[a,d][7]annulene hydrochloride ((+)-MK-801-OctaEGME x HCl).

FIG. 10 is a graph showing the ¹H NMR spectrum of (5S,10R)-12-(2,5,8,11,14,17,20,23,26,29-decaoxahentriacontan-31-yl)-5-methyl-10,11-dihydro-5H-5,10-epiminodibenzo[a,d][7]annulene hydrochloride ((+)-MK-801-DecaEGME x HCl).

FIG. 11 is a graph showing the ¹³C NMR spectrum of (5S,10R)-12-(2,5,8,11,14,17,20,23,26,29-decaoxahentriacontan-31-yl)-5-methyl-10,11-dihydro-5H-5,10-epiminodibenzo[a,d][7]annulene hydrochloride ((+)-MK-801-DecaEGME x HCl).

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Aside from CNS receptors, NMDAR antagonists also target NMDARs located outside the CNS, with pharmacodynamic effects that have potential therapeutic value. These peripheral effects potentially therapeutic, may be offset by the CNS effects of these drugs. Therefore, targeting these peripheral NMDARs by restricting their access to the CNS is a potential novel therapeutic option that prevents potentially detrimental central nervous system effects while maintaining potentially therapeutic peripheral glutamate-system modulating effects. This objective of targeting preferentially peripheral NMDARs can be pursued with new chemical entities designed to bind NMDARs and have a modulated ability to te the cross the BBB and IB. Physiologically, the BBB protects the brain by limiting access of potentially toxic molecules. The BBB allows and regulates the passage of essential nutrients and selected substances and is effective at eliminating or decreasing the passage of many other molecules, sometime referred to as xenobiotics, regulating the rate at which many substances reach brain tissue. The BBB can also completely block the access of certain molecules to the brain.

Polymer conjugates of NMDAR antagonists may impede, decrease, or modulate the crossing of the BBB by the active molecule. Potentially therapeutic effects of NMDAR antagonists designed so they have modulated or no access to the CNS may improve the safety window of these drugs while improving their therapeutic effects, centrally and, preferentially, peripherally. Polymer onjugates of NMDAR antagonists that cannot cross the BBB or the IB or have modulated or limited BBB or IB crossing capabilities can preferentially exert peripheral actions that are potentially therapeutic while reducing or avoiding central nervous system side effects.

Although the current studies of NMDAR antagonist are mainly centered at the resolution of CNS pathologies, including psychiatric and neurodegenerative diseases, NMDAR receptors may have a role in the pathogenesis and potential treatment of other diseases, including peripheral diseases, due to the presence of these receptors on peripheral organs, including lungs, heart, GI system, GU system and annexed organs (blood vessels, liver, pancreas, ovaries, testes, kidneys and immune cells).

In summary, NMDAR antagonists also target extra-CNS receptors, thereby exerting potential therapeutic effects, including anti-inflammatory effects in macrophages (see Example 1), and the amelioration of inflammatory pulmonary diseases (see citations below), and these effects can be preferentially achieved with the uses of the novel molecules object of the present application. In addition, by avoiding or limiting access to CNS, the dose of these novel molecules can be augmented to increase peripheral efficacy without causing dissociative effects mediated by binding to CNS receptors.

In order to best benefit from extra-CNS effect and/or avoid CNS effects, NMDAR antagonists can be modified by the covalent conjugation of polymers, such as for example polyethylene glycol (PEG, PEGylation) and other polymers so to modulate or impede BBB crossing, according to the size and the characteristics of the polymer chain.

This application aims at obtaining novel molecules with different degrees of affinity for the NMDAR, depreferentially targeting peripheral (i.e., extra-CNS) receptors for the treatment of diseases, disorders and conditions linked to unbalanced activity of peripheral glutamate receptors, preferentially.

Furthermore, the activity of these novel drugs at NMDARs in the CNS may not be completely abolished by polymer conjugation of the drug, but may simply be modulated, and polymer conjugation of these drugs may thus result in a more favorable pharmacodynamic or pharmacokinetic profile at both central and peripheral receptors. For example, PEGylated-based platforms can also be exploited to optimize and enhance the brain delivery of molecules characterized by a poor BBB penetration (Lu W, Zhang Y, Tan Y Z, Hu K L, Jiang X G, Fu S K. Cationic albumin-conjugated pegylated nanoparticles as novel drug carrier for brain delivery. J Control Release. 2005; 107:428-48.). Thus, these molecules may result in therapeutic effects for both CNS and extra-CNS conditions that represent an improvement in the efficacy/safety ratio of the parent molecule.

To that end, aspects of the present invention are directed to NMDAR antagonist polymer conjugates having a general structure D-(X-Poly-T)n, wherein D is a CNS active NMDAR antagonist and n is an integer comprised between 1 and 6.

X is a stable (enzymatically and/or hydrolytically under physiological conditions) linker comprising a covalent bond or a chain of atoms that covalently attaches a small molecule NMDA antagonist drug moiety to the Poly derivative. The drug moiety has at least one chemically reactive functional group (e.g. a primary amine or secondary amine, hydroxyl, sulfhydryl, carboxyl, aldehyde or ketone), or if absent this group can be chemically introduced, pendant thereto chemically reacted to the linker to form a covalent bond. Examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, thioether.

Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, mPEG-O-559 Da, where “m” means methoxy.

T, if present, is either D or a terminal group of the Poly, when T is a terminal group it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, Nsuccinimidyloxy, sulfo-N-succinimidyloxy, 1-benzotriazol, 1-imidazolyloxy, p-nitrophenyloxy, formyl. We have begun synthesizing PEG derivatives of esmethadone by connecting a short PEG chain (oligo(ethylene glycol)) to one of the phenyl functionalities of esmethadone. We selected the phenyl rings for PEG conjugation because of our previous work on esmethadone analogues suggesting that modifications at the level of the phenyl ring caused the least hindrance to NMDAR activity.

In one particular embodiment, the present invention includes a compound including a structural analogue to (R)-methadone ((−)-methadone, levomethadone), in free base form, and/or in a pharmaceutical acceptable salt form thereof according to formula I:

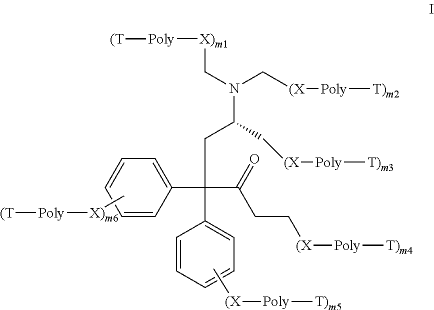

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (−)-methadone to the Poly derivative [examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether];
Poly is a covalently bonded chain of repeating monomer units that form a polymer backbone of synthetic or natural origin [examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides]. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. In certain embodiments, Poly may be chosen from mPEG-O-163 Da, mPEG-COO-207 Da, mPEG-O-251 Da, mPEG-O-295 Da, mPEG-O— 339 Da, mPEG-O-383 Da, mPEG-O-427 Da, mPEG-O-471 Da, mPEG-O-515 Da, and mPEG-O-559 Da, where “m” means methoxy; and
T, if present, is either (−)-methadone or a terminal group of Poly, [when T is a terminal group it is represented by any suitable chemical group which, depending upon preference, is unreactive or reactive with other chemical moieties; examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl].

In another particular embodiment, the present invention includes a compound including a structural analogue to (S)-methadone ((+)-methadone, dextromethadone, esmethadone), in free base form, and/or in a pharmaceutical acceptable salt form thereof, according to formula II:

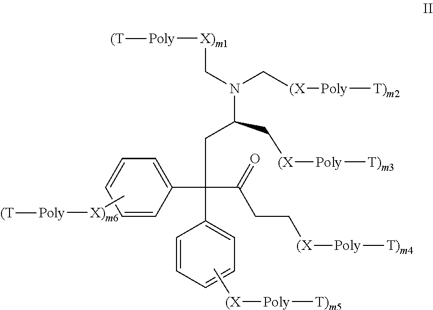

wherein m1, m2, m3, m4, m5 and m6 are, independently for each other, 0 or 1, and m1+m2+m3+m4+m5+m6 is between 1 and 6, so that at least one X-Poly-T is present;
X is a stable (enzymatically and/or hydrolytically stable under physiological conditions) linker comprising a covalent bond or a chain of atoms that covalently attaches (+)-methadone to the Poly derivative [examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether];
Poly is a covalently bonded chain of repeating monomer units that form a polymer backbone of synthetic or natural origin [examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides]. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. In certain embodiments, Poly may be mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, or mPEG-O-559 Da, where “m” means methoxy; and
T, if present, is either (+)-methadone or a terminal group of Poly, when T is a terminal group it is represented by any suitable chemical group which, depending upon preference, is unreactive or reactive with other chemical moieties [examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl].

In another particular embodiment, the present invention includes a compound having a structural analogue to (S, R)-methadone ((±)-methadone, rac-methadone, methadone), in free base form, and/or in pharmaceutical acceptable salt form thereof according to formula III:

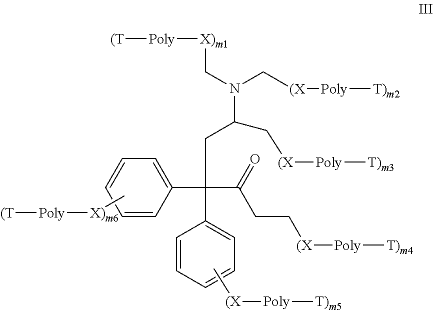

wherein m1, m2, m3, m4, m5 and m6 are, independently for each other, 0 or 1, and m1+m2+m3+m4+m5+m6 is comprised between 1 and 6, so at least one X-Poly-T is present;
X is a stable (enzymatically and/or hydrolytically under physiological conditions) linker comprising a covalent bond or a chain of atoms that covalently attaches (±)-methadone to the Poly derivative (examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether);
Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly that may be suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, and mPEG-O-559 Da, where “m” means methoxy; and
T, if present, is either (±)-methadone or a terminal group of Poly—and when T is a terminal group, it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.

In another particular embodiment, the present invention includes a compound having a structural analogue to (−)-dizocilpine ((−)-MK-801) in free base form, and/or in pharmaceutical acceptable salt form thereof according to formula IV:

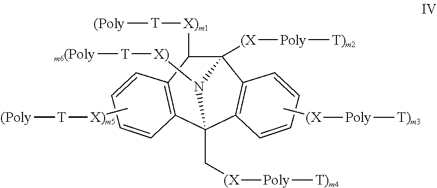

wherein m1, m2, m3, m4, m5 and m6 are, independently for each other, 0 or 1, and m1+m2+m3+m4+m5+m6 is comprised between 1 and 6, so at least one X-Poly-T is present;
X is a stable (enzymatically and/or hydrolytically stable under physiological conditions) linker comprising a covalent bond or a chain of atoms that covalently attaches (−)-dizocilpine to the Poly derivative. Examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether;
Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, and mPEG-O-559 Da, where “m” means methoxy; and
T, if present, is either (−)-dizocilpine or a terminal group of Poly—and when T is a terminal group, it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.

In another particular embodiment, the present invention includes a compound having a structural analogue to (+)-dizocilpine ((+)-MK-801) in free base form, and/or in pharmaceutical acceptable salt form thereof according to formula V:

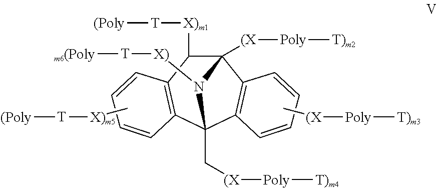

wherein m1, m2, m3, m4, m5 and m6 are, independently for each other, 0 or 1, and m1+m2+m3+m4+m5+m6 is comprised between 1 and 6, so at least one X-Poly-T is present;
X is a stable (enzymatically and/or hydrolytically stable under physiological conditions) linker comprising a covalent bond or a chain of atoms that covalently attaches (+)-dizocilpine to the Poly derivative. Examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether;
Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, and mPEG-O-559 Da, where “m” means methoxy; and
T, if present, is either (+)-dizocilpine or a terminal group of Poly—and when T is a terminal group it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.

In another particular embodiment, the present invention includes a compound having a structural analogue to (±)-dizocilpine ((±)-MK-801) in free base form, and/or in pharmaceutical acceptable salt form thereof according to formula VI:

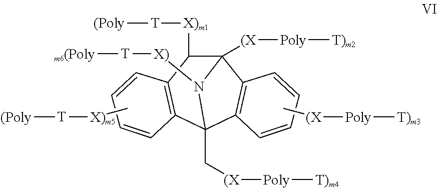

wherein m1, m2, m3, m4, m5 and m6 are, independently for each other, 0 or 1, and m1+m2+m3+m4+m5+m6 is comprised between 1 and 6, so at least one X-Poly-T is present;
X is a stable (enzymatically and/or hydrolytically stable under physiological conditions) linker comprising a covalent bond or a chain of atoms that covalently attaches (±)-dizocilpine to the Poly derivative. Examples of linkers include but are not limited to the following: carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether;
Poly is a covalently bonded chain of repeating monomer units that form a polymer backbone of synthetic or natural origin. Examples of polymer backbones include but are not limited to the following: poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, or polysialic acid or other polysaccharides. In certain embodiments, the polymer Poly has an average molecular weight between 80 and 40000 Da. In some embodiments, this average molecular weight is at least 100 Da. In some embodiments, this average molecular weight is at least 200 Da. In some embodiments of the invention, Poly is a derivative of poly(ethylene glycol) (PEG), of linear or branched structure, mono-, bi-functional or heterobifunctional, with an average molecular weight between 120 and 40000 Da. Some Poly suitable for the present invention include mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, and mPEG-O-559 Da, where “m” means methoxy; and
T, if present, is either (±)-dizocilpine or a terminal group of Poly—and when T is a terminal group it is represented by any suitable chemical group which, depending upon preference, is unreactive or reactive with other chemical moieties. Examples of terminal groups include but are not limited to the following: hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.

In some cases, these molecules may have reduced/abolished intestinal absorption according to the size/feature of the coupled polymeric chain, and these molecules will preferentially target intestinal receptors, being useful for the treatment of diseases og the GI tract, including inflammatory diseases of the GI tract, such as inflammatory bowel diseases, including ulcerative colitis and Chron's disease, and for the treatment of irritable bowel syndrome.

On the basis of desired therapeutic effects, it could be desired to maintain some degree of central nervous system NMDAR antagonism, or alternatively one could to generate increasingly selective peripheral modulators of NMDARs, even with no CNS activity. The BBB penetration may be modified, and the intestinal penetration may be modified to achieve the desired therapeutic use of these novel molecules.

Diseases and disorders where inflammation concurs in triggering or maintaining the pathological process are of relevance. In regard to the respiratory system, COVID-19 and other infections, long-COVID-19, inflammatory bowel disease, ARDS, and inflammatory pulmonary diseases, including asthma, chronic obstructive pulmonary disease (COPD), and pulmonary fibrosis are some examples of such diseases. Other examples of diseases, disorders, conditions and symptoms that could be treated with the molecules disclosed in this application are described in the article by Du and colleagues (Du J, Li X H, Li Y J. Glutamate in peripheral organs: Biology and pharmacology. Eur J Pharmacol. 2016; 784:42-48. doi:10.1016/j.ejphar.2016.05.009.) As also discussed above, alterations in NMDAR signaling have been described in inflammatory conditions of the respiratory system. The expression of NMDARs have been found in lung tissue. Glutamate-induced NMDAR activation may cause acute lung injury with acute pulmonary edema and promote pulmonary vascular remodeling in pulmonary hypertension (Said et al., Excitoxicity in the lung: N-methyl-D-aspartate-induced, nitric oxide-dependent, pulmonary edema is attenuated by vasoactive intestinal peptide and by inhibitors of poly (ADP-ribose) polymerase, Proc. Natl. Acad. Sci. USA 93: 4688-4692, 1996; Dickman et al. Ionotropic glutamate receptors in lungs and airways: molecular basis for glutamate toxicity, Am. J. Respir. Cell Mol. Biol. 30: 139-144, 2004; Dumas et al., NMDA-type glutamate receptor activation promotes vascular remodeling and pulmonary arterial hypertension, Circulation 137: 2371-2389, 2018). Accordingly, research has shown that NMDAR antagonists with high affinity, such as MK-801, can attenuate oxidative stress in acute lung injury induced by intratracheal lipopolysaccharide (LPS) injection (da Cunha et al., Treatment with N-methyl-D-aspartate receptor antagonist MK-801 protects against oxidative stress in lipopolysaccharide-induced acute lung injury in the rat, Int. Immunopharmacol. 11: 706-711, 2011). Notably, the noncompetitive NMDAR antagonist memantine alleviates acute lung injury (ALI) by acting on macrophages present in the respiratory tract (Ding et al., Memantine alleviates acute lung injury via inhibiting macrophage pyroptosis, Shock 56: 1040-1048, 2021) and septic lung injury (Hu et al., Memantine nitrate MN-08 suppresses NLRP3 inflammasome activation to protect against sepsis-induced acute lung injury in mice. Biomed and Pharmacother 156: 113804, 2022).

Furthermore, the inhibition of NMDARs by memantine has also been shown to be effective against chronic obstructive pulmonary disease (COPD) (Cheng et al., Memantine ameliorates pulmonary inflammation in a mice model of COPD induced by cigarette smoke combined with LPS. Biomed and Pharmacother 109: 2005-2013, 2019). This is of relevance since COPD affects more than 15 million adults in the United States, where this disease is a major cause of disability. COPD, which is strongly related to cigarette smoking, is the fourth leading cause of death in the United States according to the Centers for Disease Control and Prevention (CDC). Current therapy for COPD is relatively ineffective since the available drugs could not considerably delay the disease progression or have a substantial effect on inflammation. Hence, suppressing inflammatory responses by acting on macrophages present in the airways is considered an essential strategy for COPD treatment. Taken together, these observations point at NMDARs as potential pharmacological agents for therapeutic intervention in different diseases affecting the respiratory tract.

In accordance with these observations, our results (Example 1) suggest that NMDAR antagonists, e.g., esmethadone and MK-801 reduce macrophage-induced inflammation in pulmonary cells. In mice, MK-801 conjugated with PEG molecules of different length did not cause the behavioral alterations observed for unconjugated MK-801 (Example 2), suggesting a PEG-related modulation of BBB crossing and or other potential mechanisms. Based on our and the previously described experimental findings, NMDAR antagnonists may have strong therapeutic potential for the treatment of diseases, disorders, conditions and symptoms triggered by dysregulated peripheral NMDAR, including NMDAR that may be dysregulated in inflammatory lung diseases. The present inventors demonstrated that in vitro esmethadone and MK-801 reduce the mRNA expression of inflammatory cytokines in pulmonary cells co-cultured with macrophages. Based on these studies, the present inventors concluded that targeting inflammatory peripheral diseases including lung diseases with NMDAR antagonists with preferential peripheral actions may be a promising strategy. With the same rationale, these same polymer conjugates are potentially therapeutic for all diseases, disorders, conditions and symptoms caused by dysregulation of NMDARs on extra CNS cells, including cells part of the respiratory system, digestive system, cardiovascular system, immune system, renal system, reproductive system.

### EXAMPLES

### Example 1: NMDAR Antagonists Reduce Inflammation In Vitro

### In Vitro Study

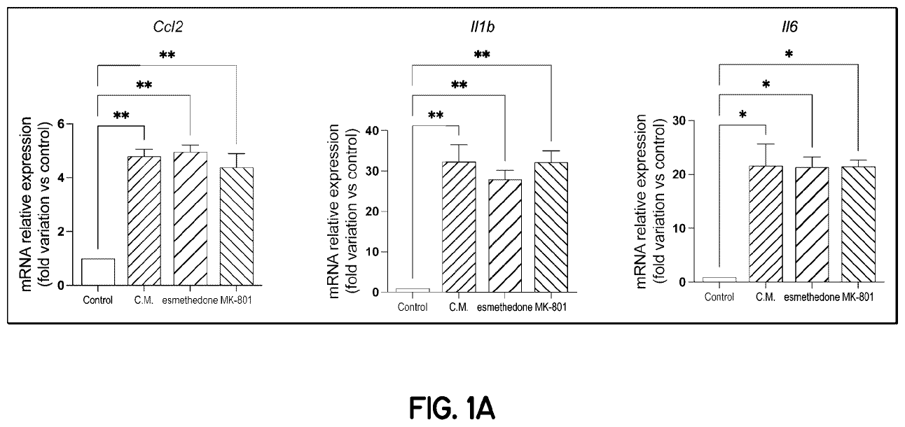

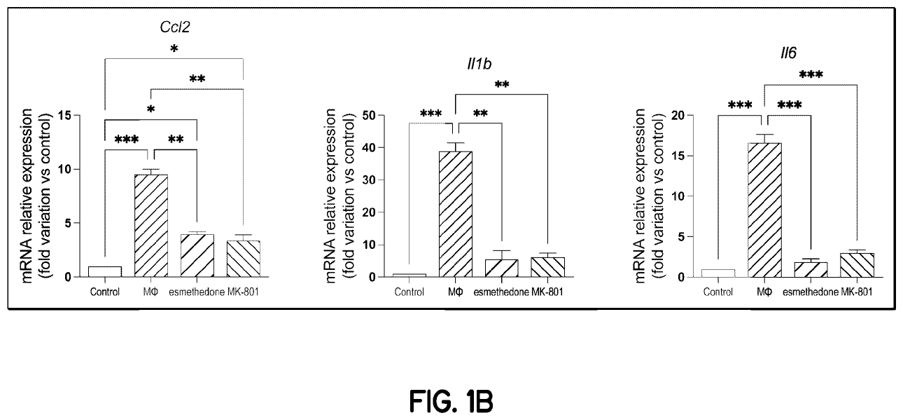

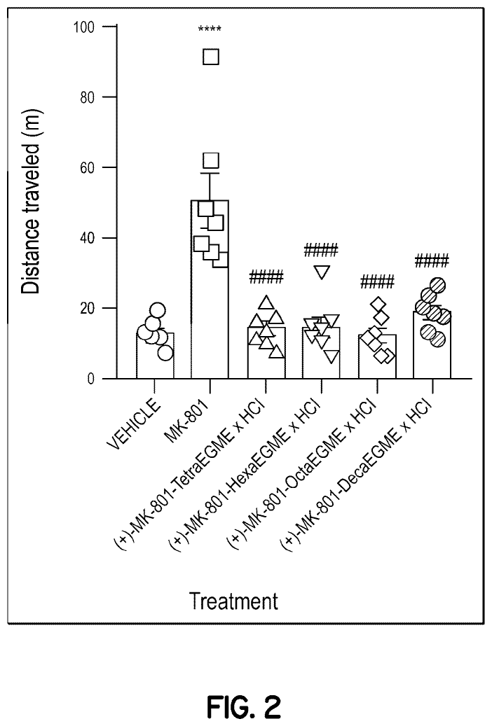

The present inventors used the A-549 lung cell line to set up an experimental in vitro model of inflammatory pulmonary disease, obtained by co-colturing these cells for 24 hours with macrophages obtained by differentiating the monocytic cells THP-1 (FIG. 1A). FIG. 1B show that esmethadone and MK-80 reduces the mRNA expression of the 2 inflammatory cytokines CCL-2 and IL-1β, only when A-549 cells were incubated with macrophages, demonstrating the pivotal role of immune cells in the mechanism of NMDAR antagonists. Thus, the anti-inflammatory effect of the NMDAR antagonists MK-801 and esmethadone (1 μM) is due to their direct interaction with macrophage-like cells [as shown by the data in FIGS. 1A and 1B—in FIG. 1A, mRNA expression of the inflammatory cytokines CCL2, IL-1b and IL-6 in pulmonary A549 cells activated by the conditioned medium obtained from macrophages (C.M.); and in FIG. 1B, mRNA expression of the inflammatory cytokines CCL2, IL-1b and IL-6 in pulmonary A549 cells in co-culture with macrophages derived by treating THP-1 cells with the activating agent PMA (MΦ)].

### In Vivo Study

The present inventors used 7 C57BL6/J male (3-4 months old) mice per treatment group, keeping them in a temperature-controlled room (22° C.) on a 12:12 h light-dark cycle (light on at 7:00 AM) and fed a standard pellet diet and tap water ad libitum. Experimental mice were habituated to the testing room by transferring them to the behavioural room 30 min prior to beginning of the test. The open field test was conducted between 9:00 am and 4:00 pm. MK-801 and derivatives were dissolved in a vehicle composed by 0.9% NaCl physiological solution. Each mouse was singly placed at the corner of a grey-painted open field arena (40×40×30 cm) 10 min after the intraperitoneal injection (Volume of injection 0.1 mL) of either Vehicle, 0.3 mg/kg MK-801, or the equivalent dose of 0.3 mg/kg MK-801 of the PEG 4 MK-801, PEG 6 MK-801, PEG 8 MK-801 or PEG 10 MK-801 derivatives. Using an automated behavioral tracking system (Videotrack, View Point Life Science), we determined the total locomotor activity measured as distance travelled by the animal during the 10 min duration of the test.

The present inventors demonstrated that the intraperitoneal injection of 0.3 mg/kg MK-801 significantly increased the distance travelled by the mice compared with that of vehicle but also of the MK-801 PEG derivatives (p<0.0001; One-way ANOVA: F (5, 36)=16,40; p<0,0001). Interestingly, all the tested PEG derivatives of MK-801 did not alter the distance travelled by the mice compared with vehicle, suggesting that PEG-derivatives did not cross the BBB.

The present inventors are here disclosing conjugates of NMDAR antagonists with modulating actions and/or other actions preferentially at extra CNS receptors. The present inventors are also disclosing the adopting polymers which can modulate/reduce/eliminate the ability of the parent drug to cross the BBB and or the intestinal barrier. The present inventors are developing molecules that can be administered, preferably but not exclusively, via the oral pathway or the pulmonary route of administration to reach their site of action. Oral administration is one of the preferred routes of administration and is the most common route of administration for small molecule drugs. The polymer chain length and features can be modulated to maintain the desired intestinal absorption for the targeted disease, disorder or condition. The desired therapeutic activity may be restricted to the gastrointestinal tract and therefore gastrointestinal (GI) crossing is restricted or completely blocked.

### Example 3: (5S,10R)-5-methyl-12-(2,5,8,11-tetraoxatridecan-13-yl)-10,11-dihydro-5H-5,10-epiminodibenzo[a,d][7]annulene hydrochloride ((+)-MK-801-TetraEGME x HCl)

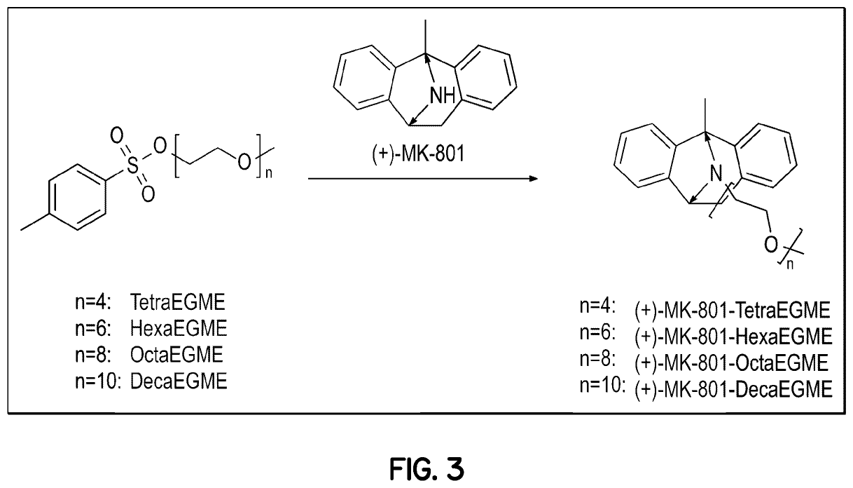

This Example 3 describes a method that was used to prepare (5S,10R)-5-methyl-12-(2,5,8,11-tetraoxatridecan-13-yl)-10,11-dihydro-5H-5,10-epiminodibenzo[a,d][7]annulene hydrochloride ((+)-MK-801-TetraEGME x HCl), as shown in FIG. 3.

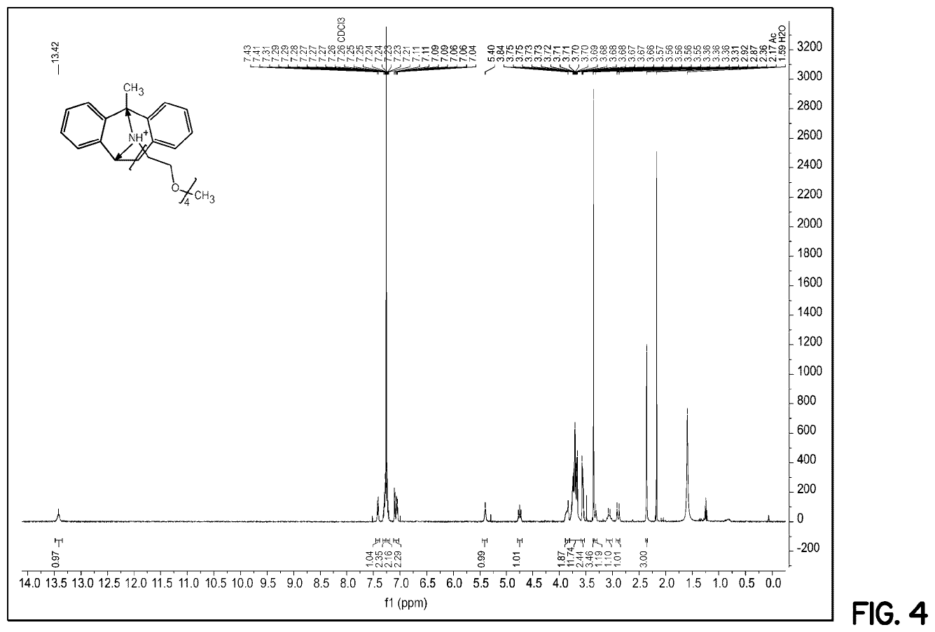

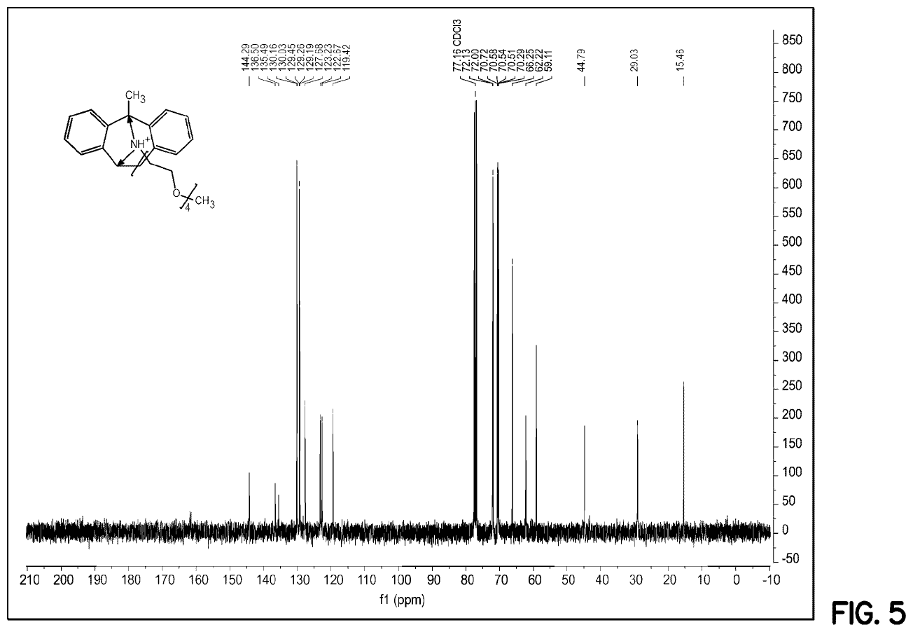

To a stirred solution of (+)-MK-801 (0.100 g, 0.45 mmol, 1 eq.) in anhydrous DMF (1.356 mL) was added Cs₂CO₃ (0.162 g, 0.50 mmol, 1.1 eq.) and KI (8 mg, 0.05 mmol, 0.1 eq.), then a solution of 2,5,8,11-tetraoxatridecan-13-yl 4-methylbenzenesulfonate (TetraEGME, 0.246 g, 0.68 mmol, 1.5 eq.) in anhydrous DMF (0.452 mL). The mixture was heated to 70° C. and stirred for 24 h under nitrogen. Then, the mixture was diluted with water (1.356 mL) and the residual carbonate was quenched with acetic acid (0.028 mL, 1.1 eq.). The mixture was purified via preparative RP-HPLC C-18 (eluent water+0.1% TFA/ACN, starting from 5% ACN and reaching 50% in 17 minutes, retention time 16.09 minutes). After freeze-drying, the obtained TFA salt was redissolved in 30% ACN in water and the trifluoroacetate counterion was exchanged using Amberlite IRA400. After freeze-drying, (+)-MK-801-TetraEGME x HCl was obtained as a colorless oil (0.1850 g, 0.353 mmol, 78%). UPLC purity: >99%. HRMS (ESI) m/z: [M+H]⁺ required [C₂₅H₃₄NO₄]⁺ 412.2482; detected: 412.2543. Specific optical rotation [α]_{D}²⁵: +88.7° (0.02 g/mL, CHCl₃). ¹H NMR (400 MHz, CDCl₃) δ 13.42 (s, 1H), 7.42 (d, J=6.9 Hz, 1H), 7.33-7.27 (m, 2H), 7.25-7.19 (m, 2H), 7.13-7.02 (m, 2H), 5.40 (s, 1H), 4.75 (t, J=10.3 Hz, 1H), 3.84 (s, 2H), 3.80-3.60 (m, 11H), 3.59-3.51 (m, 2H), 3.39-3.33 (m, 3H), 3.31 (s, 1H), 3.06 (d, J=12.4 Hz, 1H), 2.94-2.84 (m, 1H), 2.36 (s, 3H)—See FIG. 4. ¹³C NMR (101 MHz, CDCl₃) δ 144.29, 136.50, 135.49, 130.16, 130.03, 129.45, 129.26, 129.19, 127.68, 123.23, 122.67, 119.42, 72.13, 72.00, 70.72, 70.58, 70.54, 70.51, 70.29, 66.25, 62.22, 59.11, 44.79, 29.03, 15.46—See FIG. 5.

### Example 4: (5S,10R)-5-methyl-12-(2,5,8,11,14,17-hexaoxanonadecan-19-yl)-10,11-dihydro-5H-5,10-epiminodibenzo[a,d][7]annulene hydrochloride ((+)-MK-801-HexaEGME x HCl)

This Example 4 describes a method that was used to prepare (5S,10R)-5-methyl-12-(2,5,8,11,14,17-hexaoxanonadecan-19-yl)-10,11-dihydro-5H-5,10-epiminodibenzo[a,d][7]annulene hydrochloride ((+)-MK-801-HexaEGME x HCl), as shown in FIG. 3.

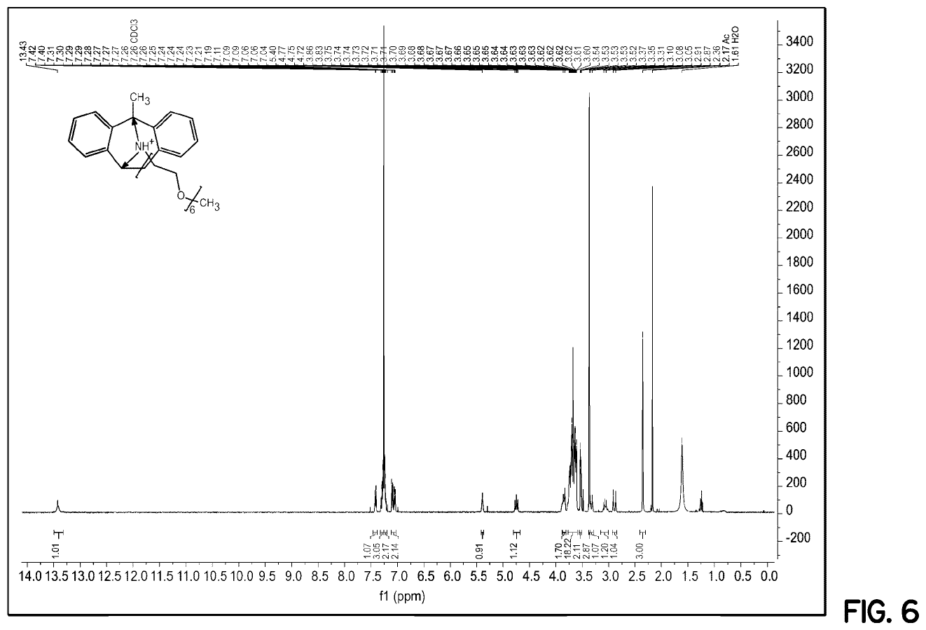

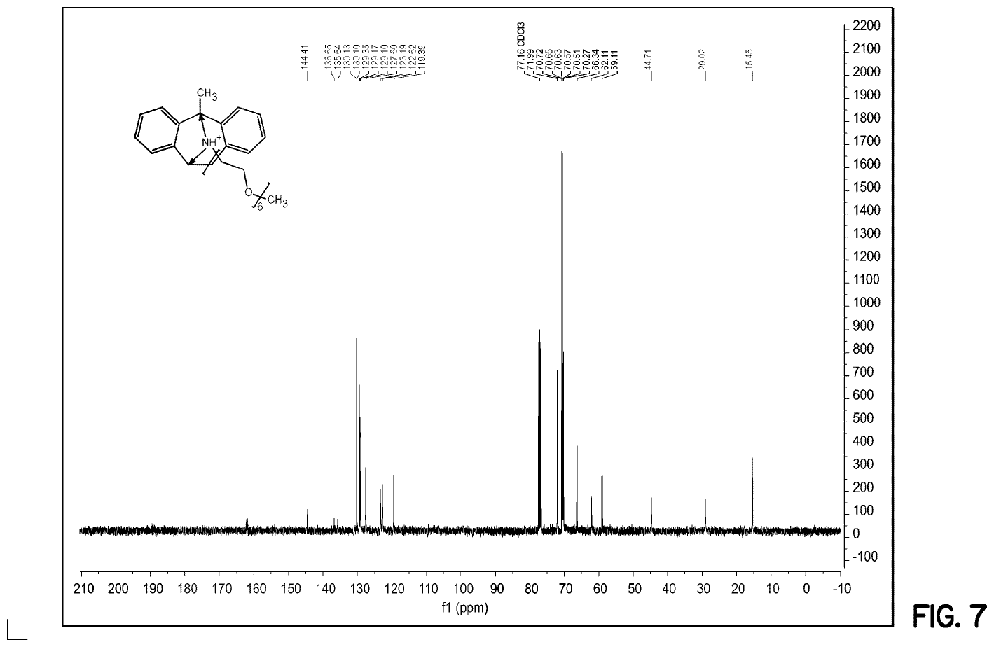

To a stirred solution of (+)-MK-801 (0.100 g, 0.45 mmol, 1 eq.) in anhydrous DMF (1.356 mL) was added Cs2CO3 (0.162 g, 0.50 mmol, 1.1 eq.) and KI (8 mg, 0.05 mmol, 0.1 eq.), then a solution of 2,5,8,11,14,17-hexaoxanonadecan-19-yl 4-methylbenzenesulfonate (HexaEGME, 0.305 g, 0.68 mmol, 1.5 eq.) in anhydrous DMF (0.452 mL). The mixture was heated to 70° C. and stirred for 24 h under nitrogen. Then, DMF was diluted with water (1.356 mL) and the residual carbonate was quenched with acetic acid (0.028 mL, 1.1 eq.). The mixture was purified via preparative RP-HPLC C-18 (eluent water+0.1% TFA/ACN, starting from 5% ACN and reaching 50% in 17 minutes, retention time 16.65 minutes). After freeze-drying, the obtained TEA salt was redissolved in 30% ACN in water and the trifluoroacetate counterion was exchanged using Amberlite IRA400. After freeze-drying, (+)-MK-801-HexaEGME x HCl was obtained as a colorless oil (0.239 g, 0.39 mmol, yield 87%). UPLC purity: >95%. HRMS (ESI) m/z: [M+H]⁺ required [C₂₉H₄₂NO₆]⁺: 500.3007; detected: 500.3035. Specific optical rotation [α]_{D}²⁵: +81.93 (0.02 g/mL, CHCl3). ¹H NMR (400 MHz, CDCl3) δ 13.43 (s, 1H), 7.45-7.38 (m, 1H), 7.34-7.24 (m, 3H), 7.27-7.17 (m, 2H), 7.13-7.02 (m, 2H), 5.40 (s, 1H), 4.75 (t, J=10.5 Hz, 1H), 3.85 (d, J=11.6 Hz, 2H), 3.77-3.58 (m, 18H), 3.56-3.49 (m, 2H), 3.37 (s, 3H), 3.33 (d, J=15.6 Hz, 1H), 3.07 (t, J=11.1 Hz, 1H), 2.93-2.83 (m, 1H), 2.36 (s, 3H)—See FIG. 6. ¹³C NMR (101 MHz, CDCl3) δ 144.41, 136.65, 135.64, 130.13, 130.10, 129.35, 129.17, 129.10, 127.60, 123.19, 122.62, 119.39, 71.99, 70.72, 70.65, 70.63, 70.57, 70.51, 70.27, 66.34, 62.11, 59.11, 44.71, 29.02, 15.45—See FIG. 7.

### Example 5: (5S,10R)-5-methyl-12-(2,5,8,11,14,17,20,23-octaoxapentacosan-25-yl)-10,11-dihydro-5H-5,10-epiminodibenzo[a,d][7]annulene hydrochloride ((+)-MK-801-OctaEGME x HCl)

This Example 5 describes a method that was used to prepare (5S,10R)-5-methyl-12-(2,5,8,11,14,17,20,23-octaoxapentacosan-25-yl)-10,11-dihydro-5H-5,10-epiminodibenzo[a,d][7]annulene hydrochloride ((+)-MK-801-OctaEGME x HCl), as shown in FIG. 3.

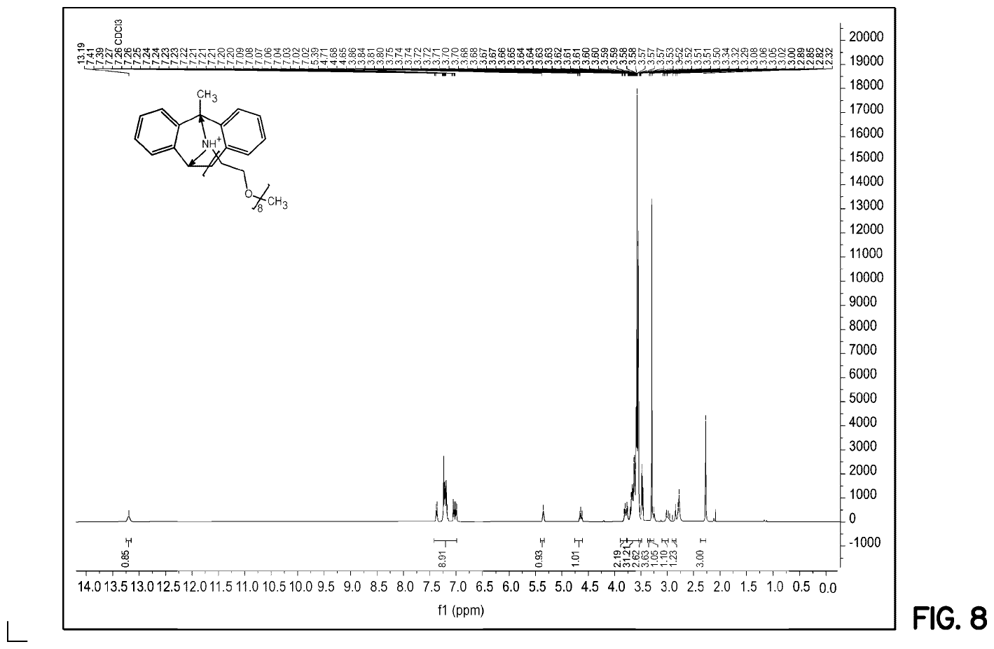

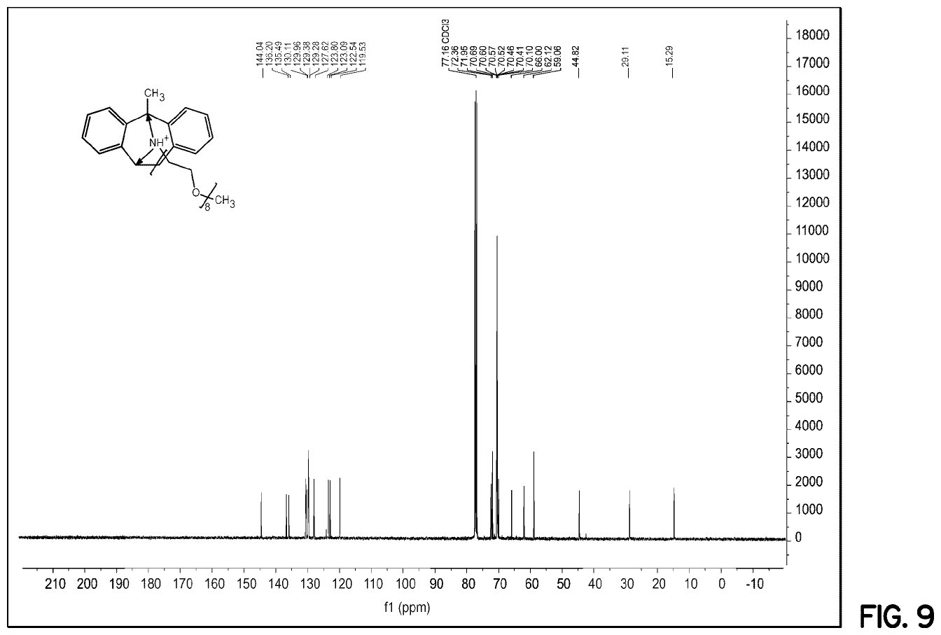

To a stirred solution of (+)-MK-801 (0.100 g, 0.45 mmol, 1 eq.) in anhydrous DMF (1.35 mL) was added Cs₂CO₃ (0.162 g, 0.50 mmol, 1.1 eq.) and KI (8 mg, 0.05 mmol, 0.1 eq.), then a solution of 2,5,8,11,14,17,20,23-octaoxapentacosan-25-yl 4-methylbenzenesulfonate (OctaEGME, 0.365 g, 0.68 mmol, 1.5 eq.) in anhydrous DMF (0.45 mL). The mixture was heated to 80° C. and stirred for 24 h under nitrogen. Then, DMF was diluted with water (2 mL) and the residual carbonate was quenched with acetic acid (0.028 mL, 1.1 eq.). The mixture was purified via preparative RP-HPLC C-18 (eluent water+0.1% TFA/ACN, starting from 5% ACN and reaching 56% in 19 minutes, retention time 17.55 minutes). After freeze-drying, the obtained TFA salt was redissolved in 30% ACN in water and the trifluoroacetate counterion was exchanged using Amberlite IRA400. After freeze-drying, (+)-MK-801-OctaEGME x HCl was obtained as a colorless oil (0.2571 g, 0.37 mmol, 82% yield). UPLC purity: >99%. HRMS (ESI) m/z: [M+H]⁺ required [C₃₃H₅₀NO₈]⁺: 588.3531; detected: 588.3586. Specific optical rotation [α]_{D}²⁵: +61.96° (0.019 g/mL, CHCl₃). ¹H NMR (400 MHz, CDCl₃) δ 13.19 (s, 1H), 7.47-6.93 (m, 8H), 5.39 (s, 1H), 4.68 (t, J=10.4 Hz, 1H), 3.88-3.78 (m, 2H), 3.78-3.55 (m, 26H), 3.54-3.48 (m, 2H), 3.34 (s, 3H), 3.31 (d, J=13.2 Hz, 1H), 3.10-2.98 (m, 1H), 2.91-2.84 (m, 1H), 2.32 (s, 3H)—See FIG. 8. ¹³C NMR (101 MHz, CDCl₃) δ 144.04, 136.20, 135.49, 130.11, 129.96, 129.38, 129.28, 127.62, 123.80, 123.09, 122.54, 119.53, 72.36, 71.95, 70.69, 70.60, 70.57, 70.52, 70.46, 70.41, 70.10, 66.00, 62.12, 59.06, 44.82, 29.11, 15.29—See FIG. 9.

### Example 6: (5S,10R)-12-(2,5,8,11,14,17,20,23,26,29-decaoxahentriacontan-31-yl)-5-methyl-10,11-dihydro-5H-5,10-epiminodibenzo[a,d][7]annulene hydrochloride ((+)-MK-801-DecaEGME x HCl)

This Example 6 describes a method that was used to prepare (5S,10R)-12-(2,5,8,11,14,17,20,23,26,29-decaoxahentriacontan-31-yl)-5-methyl-10,11-dihydro-5H-5,10-epiminodibenzo[a,d][7]annulene hydrochloride ((+)-MK-801-DecaEGME x HCl), as shown in FIG. 3.

To a stirred solution of (+)-MK-801 (0.016 g, 0.07 mmol, 1 eq.) in anhydrous DMF (0.217 mL) was added Cs₂CO₃ (0.026 g, 0.08 mmol, 1.1 eq.) and KI (1 mg, 0.01 mmol, 0.1 eq.), then a solution of 2,5,8,11,14,17,20,23,26,29-decaoxahentriacontan-31-yl 4-methylbenzenesulfonate (DecaEGME, 0.068 g, 0.11 mmol, 1.5 eq.) in anhydrous DMF (0.072 mL). The mixture was heated to 80° C. and stirred for 24 h under nitrogen. Then, DMF was diluted with water (3 mL) and the residual carbonate was quenched with acetic acid (0.028 mL, 1.1 eq.). The mixture was purified via preparative RP-HPLC C-18 (eluent water+0.1% TFA/ACN, starting from 5% ACN and reaching 57% in 20 minutes, retention time 19.42 minutes). After freeze-drying, the obtained TFA salt was redissolved in 30% ACN in water and the trifluoroacetate counterion was exchanged using Amberlite IRA400. After freeze-drying, (+)-MK-801-DecaEGME x HCl was obtained as a colorless oil (0.039 g, 0.050 mmol, 72% yield). UPLC purity: >99%. HRMS (ESI) m/z: [M+H]⁺ required [C₃₇H₅₈NO₁₀]⁺: 676.4055; detected: 676.4047. Specific optical rotation [α]_{D}²⁵: +63.27° (0.009 g/mL, CHCl₃).

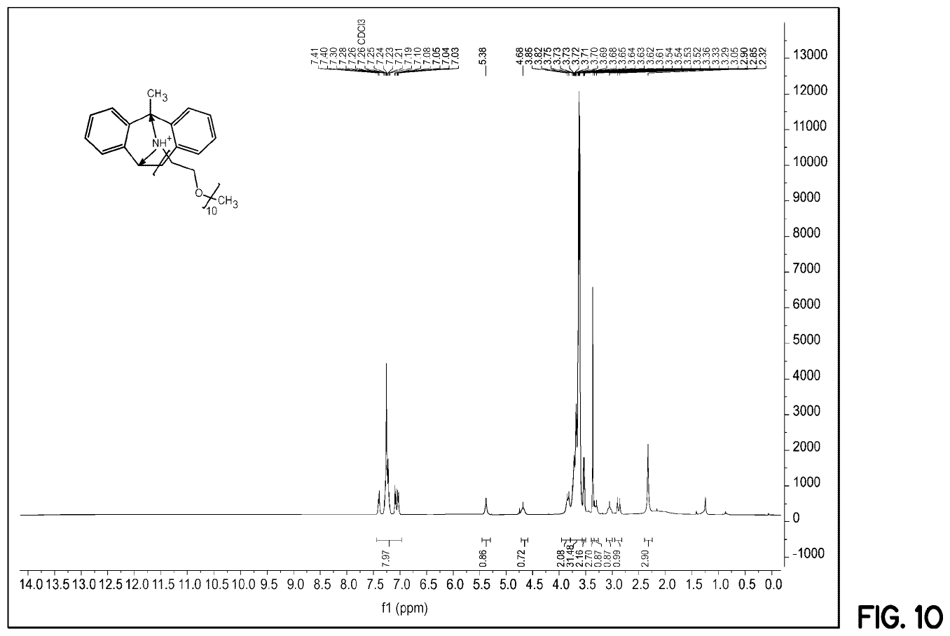

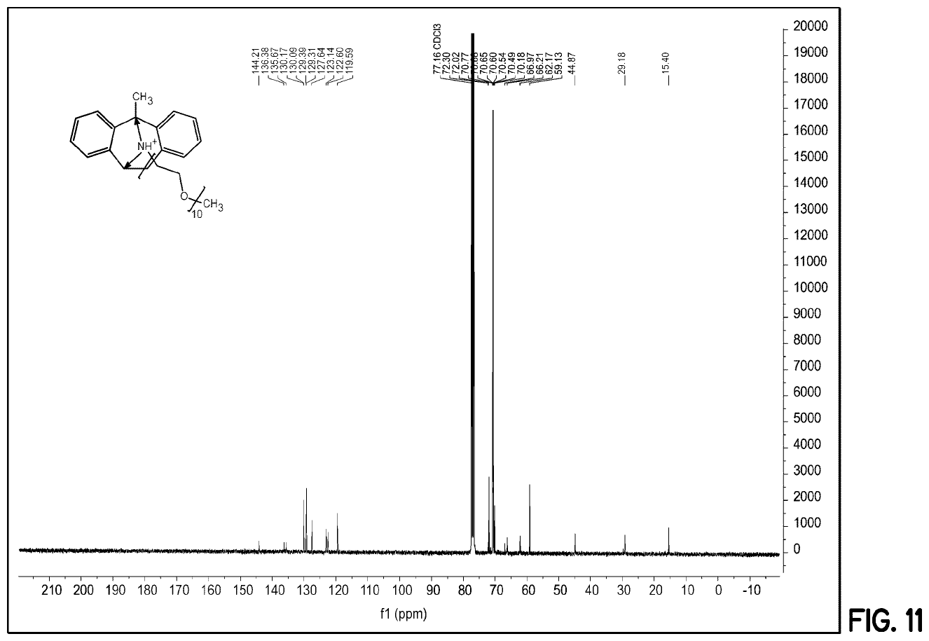

¹H NMR (400 MHz, CDCl₃) δ 7.44-6.99 (m, 8H), 5.38 (s, 1H), 4.72-4.63 (m, 1H), 3.84 (d, J=14.2 Hz, 2H), 3.78-3.57 (m, 34H), 3.57-3.50 (m, 2H), 3.36 (s, 3H), 3.31 (d, J=15.3 Hz, 1H), 3.05 (s, 1H), 2.95-2.81 (m, 1H), 2.32 (s, 3H)—See FIG. 10. ¹³C NMR (101 MHz, CDCl₃) δ 144.21, 136.38, 135.67, 130.17, 130.09, 129.39, 129.31, 127.64, 123.14, 122.60, 119.59, 72.30, 72.02, 70.77, 70.68, 70.65, 70.60, 70.54, 70.49, 70.18, 66.97, 66.21, 62.17, 59.13, 44.87, 29.18, 15.40—See. FIG. 11.

While the present invention has been disclosed by reference to the details of various embodiments of the invention, it is to be understood that the disclosure is intended as an illustrative rather than in a limiting sense, as it is contemplated that modifications will readily occur to those skilled in the art, within the spirit of the invention and the scope of the amended claims.

1. A compound having a structural analogue to (R)-methadone ((−)-methadone, levomethadone), in free base form, and in pharmaceutical acceptable salt form thereof according to formula I; 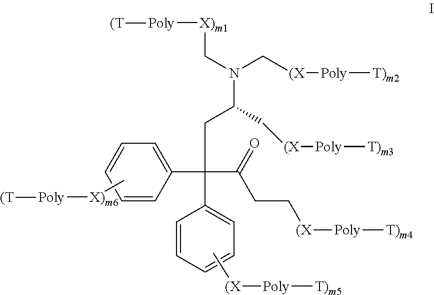
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly;
Poly is a covalently bonded chain of repeating monomer units that form a polymer backbone;
T, is optional, and is either (−)-methadone, or a terminal group of Poly.

2. A compound having a structural analogue to (S)-methadone ((+)-methadone, dextromethadone, esmethadone), in free base form, and/or in pharmaceutical acceptable salt form thereof according to formula II: 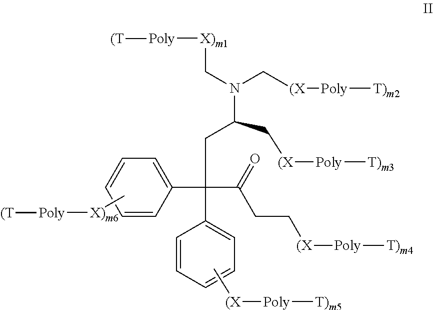
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly.
Poly is a covalently bonded chain of repeating monomer units that form a polymer backbone; and
T, is optional, and is either (−)-methadone, or a terminal group of Poly.

3. A compound having a structural analogue to (S, R)-methadone ((±)-methadone, rac-methadone, methadone), in free base form, and/or in pharmaceutical acceptable salt form thereof according to formula III: 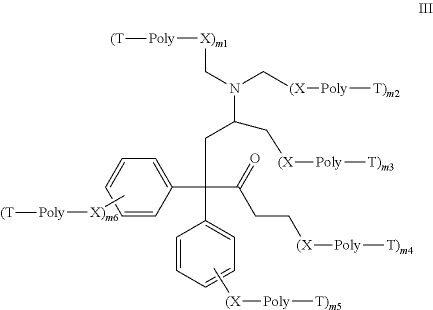
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly;
Poly is a covalently bonded chain of repeating monomer units that form a polymer backbone; and
T, is optional, and is either (−)-methadone, or a terminal group of Poly.

4. A compound having a structural analogue to (−)-dizocilpine ((−)-MK-801) in free base form, and in pharmaceutical acceptable salt form thereof according to formula IV: 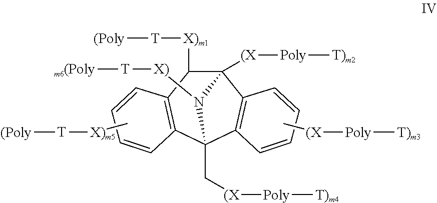
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly;
Poly is a covalently bonded chain of repeating monomer units that form a polymer backbone; and
T, is optional, and is either (−)-methadone, or a terminal group of Poly.

5. A compound having a structure analogue to (+)-dizocilpine ((+)-MK-801) in free base form, and in pharmaceutical acceptable salt form thereof according to formula V: 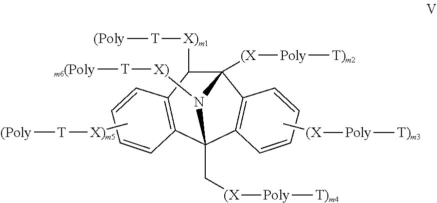
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly;
Poly is a covalently bonded chain of repeating monomer units that form a polymer backbone; and
T, is optional, and is either (−)-methadone, or a terminal group of Poly.

6. A compound having a structure analogue to (±)-dizocilpine ((±)-MK-801) in free base form, and in pharmaceutical acceptable salt form thereof according to formula VI: 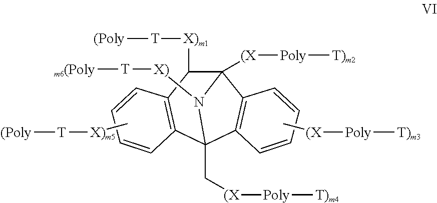
wherein
m1, m2, m3, m4, m5 and m6 are, independently, 0 or 1, and m1+m2+m3+m4+m5+m6 is between 1 and 6;
X is a stable linker comprising a covalent bond or a chain of atoms that covalently attaches (−)-methadone to Poly;
Poly is a covalently bonded chain of repeating monomer units that form a polymer backbone; and
T, is optional, and is either (−)-methadone, or a terminal group of Poly.

7. The compound of any of claims 1-6, wherein X is chosen from carboxylate ester, phosphate ester, anhydride, acetal, ketal, acyloxyalkyl ether, imine, hydrazone, carbohydrazone, carbamate, peptides, nucleotides, C—C bond (e.g., in aliphatic chain), ether, amide, oxime, enamine, semicarbazone, semicarbazide, and thioether.

8. The compound of any of claims 1-6, wherein the polymer backbone formed by Poly is chosen from poly(ethylene glycol) (PEG), poly(N-vinylpyrrolidone), N-hydroxy-ethyl methacrylamide copolymer, poly(2-ethyl-2-oxazoline), poly(N-acryloylmorpholine), poly(propylene glycol), poly(vinyl alcohol), polyglutamic acid, hyaluronic acid, polysialic acid, and other polysaccharides.

9. The compound of any of claims 1-6, wherein Poly is a derivative of poly(ethylene glycol) (PEG), of linear or branched structure, mono-, bi-functional or heterobifunctional, with an average molecular weight between 120 and 40000 Da.

10. The compound of claim 9, wherein Poly is chosen from mPEG-O-163 Da, mPEG-COO-207 Da, mPEG-O-251 Da, mPEG-O-295 Da, mPEG-O-339 Da, mPEG-O-383 Da, mPEG-O-427 Da, mPEG-O-471 Da, mPEG-O-515 Da, mPEG-O-559 Da.

11. The compound of claim 10, wherein Poly has an average molecular weight between 80 and 40000 Da.

12. The compound of claim 10, wherein Poly has an average molecular weight of at least 100 Da.

13. The compound of claim 10, wherein Poly has an average molecular weight of at least 200 Da.

14. The compound of claim 10, wherein Poly has a molecular weight greater than 500 Da and lower than 2000 Da.

15. The compound of any of claims 1-6, wherein T is a terminal group and is chosen from hydroxyl, amino, sulfide, carboxy, cyano, optionally substituted aryloxy, lower alkoxy (e.g., methoxy, ethoxy, propoxy, or butoxy), aryl, lower alkyl, lower alkenyl, lower alkynyl, cycloalkyl, halogen atom (e.g., fluorine, chlorine, bromine, iodine), tosylate, mesylate, isocyanate, hydrazine, azide, maleimide, orthopyridyl disulfide, N-succinimidyloxy, sulfo-N-succinimidyloxy, 1-benzotriazol, 1-imidazolyloxy, p-nitrophenyloxy, and formyl.

16. The compound of any of claims 1-6 having a modulated ability to cross the blood brain barrier.

17. The compound of any of claims 1-6 for the use in treating diseases affecting the peripheral cells, said peripheral cells being cells that reside outside of the blood brain barrier; or
for use in treating diseases caused by dysfunction of immune system cells, digestive system cells, respiratory system cells, cardiovascular system cells, renal system cells, reproductive system cells; or
for use in preventing diseases caused by dysfunction of immune system cells, digestive system cells, respiratory system cells, cardiovascular system cells, renal system cells, reproductive system cells; or
for the treatment or prevention of respiratory diseases, including asthma, chronic obstructive pulmonary disease (COPD), and pulmonary fibrosis, infections; or
for the treatment or prevention of cardiovascular diseases, including congestive heart failure, ischemi heart disease and arrhythmias; or
for the treatment or prevention of digestive system diseases, including Chron's disease, ulcerative colitis, irritable bowel syndrome, impaired glucose tolerance and diabetes, hepatic dysfunction; or
for the treatment or prevention of renal system diseases, including chronic and acute renal failure, renal toxicity by different substances; or
for the treatment or prevention of reproductive system diseases, including infertility.

18. (canceled)

19. (canceled)

20. (canceled)

21. (canceled)

22. (canceled)

23. (canceled)

24. (canceled)

25. A pharmaceutical or diagnostic composition comprising a compound as defined in any of claims 1-6, also comprising one or more pharmaceutically acceptable excipient.

26. The composition of claim 25 for oral, sublingual, transmucosal, intranasal, transdermal, parenteral, rectal, topical, vaginal, ophthalmic or inhalation use.

27. The composition of claim 26 administered at doses ranging from 0.001 mg to 1 gram.
